(19) United States
(12) Patent Application Publication
(10) Pub. No.: US 2004/0249250 A1
(43) Pub. Date: Dec. 9, 2004

(54) System and apparatus for monitoring and prompting medical self-care events and communicating medical self-care status

(76) Inventors: Michael D. McGee, Wenham, MA (US); Jian Shi, Shelton, CT (US)

Correspondence Address: Bourque & Associates, P.A., Suite 301, 835 Hanover Street, Manchester, NH, 03104, US

(21) Appl. No.: 10/856,625

(22) Filed: May 28, 2004

Related U.S. Application Data
(60) Provisional application No. 60/476,156, filed on Jun. 4, 2003.

(51) Int. Cl. A61B 5/00

(52) U.S. Cl. 600/300; 340/573.1

(57) ABSTRACT

A system and apparatus for monitoring self-care behavior and health status, for prompting patient self-care events such as taking medications, and for reporting self-care behavior and health status to the patient's caregivers and concerned others. The system (Self-Care Optimization System, or SOS) uses a remote data collection/messaging device (SOS device) that communicates with the SOS. The SOS monitors and promotes positive self-care behavior and collects health status data, such as blood sugar levels, blood pressure, weights, blood oxygenation levels, etc. for transmission to the SOS. The SOS prompts patients to perform self-care tasks by telephone, email, page, fax, or by sending alert messages to the SOS device. The SOS notifies medical professionals, caregivers, and/or concerned others when a pre-programmed self-care event occurs or has not occurred, of abnormal health status values such as high blood pressures, and of emergency requests for assistance made by patients pressing a “help alert” button on the SOS device.

### CROSS-REFERENCE TO RELATED APPLICATIONS

This application is related to and claims the benefit of Provisional U.S. patent application No. 60/476,156 titled SYSTEM AND APPARATUS FOR MONITORING AND PROMPTING MEDICAL SELF-CARE EVENTS AND COMMUNICATING MEDICAL SELF-CARE STATUS, filed by McGee et al. on Jun. 4, 2003, and incorporated herein by reference.

### TECHNICAL FIELD OF THE INVENTION

The present invention relates to a medical system and apparatus, and more particularly, to a system and apparatus for monitoring and reporting heath status and self-care compliance, and for prompting self-care events.

### DESCRIPTION OF THE RELATED ART

Medical doctors prescribe medications to their patients. They also give instructions for monitoring health status data such as blood pressure, blood glucose levels, oxygen levels, temperature and weight. Thereafter, the patients are responsible for taking the medications and measuring their health status. Moreover, patients must take prescribed medication doses and take health status measurements at the prescribed times. Many times, patients fail to take their medications or check their health status at the prescribed times. There are significant health concerns if a patient forgets to take his or her medication or take their health status measurements.

Physicians, Disease Management specialists and other healthcare providers increasingly rely on patient-collected health status data in order to optimize care and intervene when patients deteriorate clinically. Such data, when reported and collected real-time can help reduce morbidity, mortality and health care costs.

This risks of failures to comply with care and report abnormal health status results are greater with seriously ill or older patients. These patients may have impaired mental capacities, making them more prone to forget to take their medications or take their health status measurements. There is a need for a system or apparatus for monitoring, and prompting medical self-care events (e.g., taking medications, taking health status measurements) and communicating medical self-care status (e.g., blood glucose levels), compliance or non-compliance (e.g., patient failed to take medication at the prescribed time or failed to check their blood glucose level) to a medical provider or monitor.

It is also common to equip seriously ill or older patients with a medical alarm device. Traditionally, these devices are part of a computer system. The problem with these computer systems is that they are passive and do not offer help unless the patient explicitly asks for it. Further, the patient's commands may be different from the patient's intention. Also, the patient does not always know when he or she might benefit from asking for help. Further, a patient may be incapacitated and unable to request help via the medical alarm device.

Accordingly, there is a need for a system and apparatus for reminding the patient of a self-care event, for alerting medical professionals, caregivers, or concerned others that the patient has failed to satisfy a medical self-care event and for providing real-time health status data to caregivers.

### SUMMARY OF THE INVENTION

The present invention is a system and apparatus (hereinafter collectively “Self-Care Optimization System” or “SOS” or “System” for short) for monitoring, reminding, and alerting the patient and/or the patient's medical professional, caregiver, and/or concerned other, via messaging devices (hereinafter “SOS devices”). According to the present invention, the SOS monitors and promotes positive self-care behavior, such as compliance with medication regimens, and collects and reports health status data, such as blood sugar levels, blood pressure, weight, etc.

The SOS prompts patients to take medications, perform tests (e.g., check blood sugars), or perform other self-care tasks at specific times. The SOS improves self-care compliance with self-care tasks and provides health status data. The SOS sends real-time health status data to medical professionals, caregivers, and/or concerned others, thereby facilitating real-time intervention to address health concerns or self-care non-compliance (e.g., failure to take medication or complete other self-care tasks at prescribed time). The SOS alerts medical professionals, caregivers and/or concerned others when a patient fails to perform a scheduled self-care task and prompts the patient with a reminder, which allows for real-time outside intervention in order to improve compliance and to respond to a medical emergency.

The SOS includes a SOS device that is used by patients for: 1) sending “help” or “panic” messages, 2) transmitting health status data and 3) sending “device used” and or other messages to the SOS which, then sends alert messages to medical professionals, caregivers, and/or concerned others, 4) receiving alert messages from the SOS. A SOS device consists of one RF device and one data capture device.

Patients may use the SOS for compliance and health status monitoring, receiving reminders, sending alerts, or any combination of these functions. The SOS is an automated messaging system. Based on the patient's configuration, the SOS provides messages to the patient, medical professionals, caregivers, and/or concerned others through various communication channels such as a telephone, pager, fax, email, etc.

### BRIEF DESCRIPTION OF THE DRAWINGS

These and other features and advantages of the present invention will be better understood by reading the following detailed description, taken together with the drawings wherein:

FIG. 1 is a system diagram of a Self-care Optimization System (SOS) according to the present invention;

FIG. 2 is a schematic diagram of a wireless communication device (hereinafter “SOS device”) that includes an RF device and a data capture device.

FIG. 3 is a schematic diagram of an RF device used by an SOS device according to one embodiment of the present invention;

FIG. 4 is a schematic diagram of one network topology in which the present invention may operate;

FIG. 5 is a schematic diagram of a second alternative embodiment of a network topology for the RF device used by an SOS device of the present invention;

FIG. 6 is a schematic diagram of a third alternative embodiment of yet another network topology for the RF device used by an SOS device of the present invention;

FIG. 7 is a software architecture of the SOS according to the present invention; and

FIG. 8 is a database schema used in the SOS according to the present invention.

### DETAILED DESCRIPTION OF THE PREFERRED EMBODIMENTS

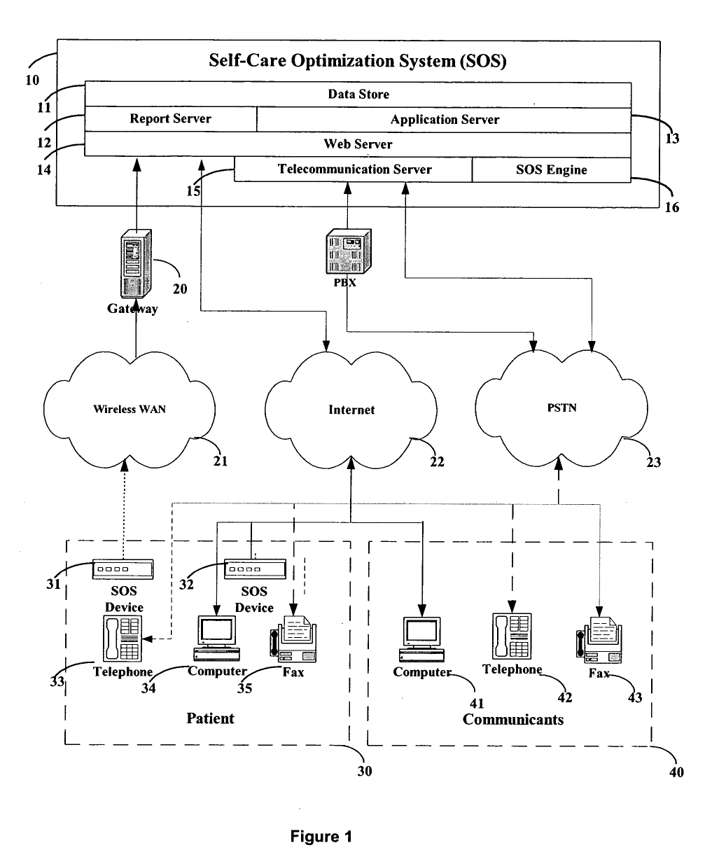

The present invention is a Self-Care Optimization System 10 (hereinafter “SOS”) shown in FIG. 1 for monitoring, messaging, and alerting of medical or other similar situations. The SOS 10 receives and sends out messages through a variety of communication channels from and to communication terminals such as a telephone 33, 42, fax 35, 43, computer email 34, 41, pager, web browser, etc. The messages are in either text or voice format. In the preferred embodiment, there is an SOS device 31 that sends an identifying signal for patient-related messages.

The user (medical professionals, caregivers, and/or concerned others) or patient (collectively referred to herein as “the user”) defines the message content or selects default messages. The user may type text messages via the Internet 22 and record voice messages via a telephone 33 or 42 in sound files in the SOS 10. Each message has its unique identification. Message identifications and their paths are stored in a database.

The user configures message recipients, messages, and communication channels for ad hoc and automated messaging. The user configures the SOS 10 to send messages triggered by SOS devices to specific message recipients via one or more specified communication channels. In the preferred embodiment, the alerting device is an SOS device 31, 32. There are one or more communication channels. If a primary communication channel is unavailable, a secondary communication channel is used. If the secondary communication is unavailable, then a tertiary communication channel is used, and so forth. Users may also compose a sequence of messages into one output notification message. Three types of events may trigger output messages: 1) time or other data parameters; 2) the user may manually trigger a message; and 3) signals from the SOS devices 31 described herein.

Outgoing messages may contain variables, such as data from other database tables (i.e., a date, a test result, a name, a telephone number, text etc). Users can create and send Ad hoc messages either individually or in batch to a specified set of message recipients based on recipient characteristics stored in the database or upon individual recipient selection from lists of available recipients.

Outgoing messages may be triggered by time parameters. Time parameters may be set for specific days of the week, month or year, specific weeks of the month, or patterns of days, weeks, or months or years (e.g. every other day, every third day, every other week, etc.) as well as for specific times of the day. Other triggering parameters may include upper and lower data thresholds (both normal and panic levels) for data retrieved from SOS devices 31.

The user specifies how outgoing messages are delivered, via one or more of the output channels described herein. The messages are generated in an appropriate XML format. The messages are then converted to channel-specific formatted messages for delivery via the specified channel. The user may send outgoing help, panic or other messages via activation of a “Help”, “panic” or other similar type button on their SOS device 31, 32. This feature may be used in an emergency as an additional alert option to the telephone or other Health Alert devices if the patient or user is in severe distress.

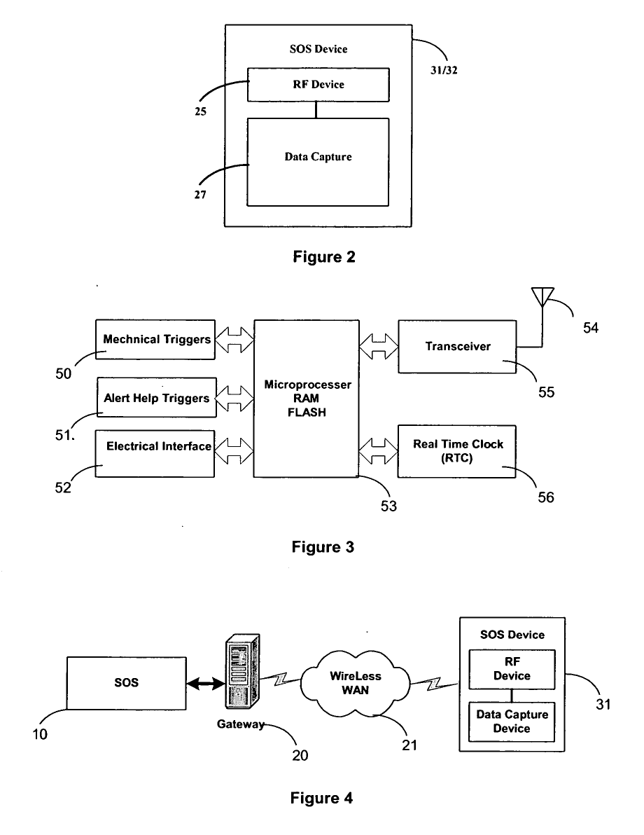

The SOS 10 has the option to use SOS devices 31 to monitor the patient's behavior and interact with the system. The SOS device 31, 32 includes an RF device and a data capture device as shown in FIG. 2. If the system senses that the patient has not performed a self-care task (e.g., taken a blood pressure reading) by not receiving expected messages from the SOS device 31, 32 at pre-specified time, the SOS initiates reminder messages to the patient or alert messages to medical professionals, caregivers, or concerned others. If the system collects health data that is out of the norm, such as too high or too low blood pressures or blood sugar levels, the system can automatically send “out of range” or “panic” messages to medical professionals, caregivers, or concerned others.

More specifically, the SOS 10 has the option to integrate with remote wireless sensors (i.e. the SOS devices) to monitor a patient's behavior, collect health data, and transmit help alerts or simply transmit that information to the SOS system for recording. The SOS 10 provides patients with help to enforce self-care tasks based on the data from these SOS devices 31, 32 combined with patient-specific self-care schedules in the SOS 10, which are input by the user of the system. The SOS devices 31, 32 use non-command interfaces to alleviate the burden of patients or others to explicitly command the SOS 10.

The SOS 10 has multiple input and output channels. The user input channels include: 1) Internet Browser; 2) Fax-scan; 3) Telephone; 4) Electronic Data input; 5) PDA; and 6) SOS device. The monitoring input channel includes SOS devices.

The output channels include: 1) Internet Browser; 2) Email; 3) Fax; 4) Telephone; 5) PDA; 6) Pager; and 7) SOS devices.

Output message triggering occurs via multiple sources and parameters, including: 1) Date and Time parameters (e.g. appointment reminders, reminders linked to medication dosage times, reminders for daily blood pressure monitoring, reminders to check blood glucose levels, etc.); 2) Ad Hoc Messaging (e.g. messages from providers to one or more of their patients, messages from administrative staff to providers or patients, individually or in batch); 3) Data Value parameters for measurement data received from SOS devices 31, 32. (e.g., alerts to clinical staff when a blood pressure or blood glucose level is above or below normal limits or beyond panic levels); 4) SOS event signals (e.g., simple notification to the SOS 10 that the device has been triggered, such as when a patient opens a medication box or uses a medical device); and 5) Patient triggering of an SOS device Help alert.

In the preferred embodiment of the SOS 10, the SOS device 31, 32 is triggered by patient activities such as opening a medicine dispenser or other container, using a medical device, or by the patient's activation of the SOS device 31, 32 Help alert. The SOS device 31, 32 sends a signal via a fixed, leased radio frequency. This signal includes an identification that uniquely identifies the device combined with device-specific data and/or Help alert data.

FIG. 1 presents a sample application environment for the SOS 10. The SOS 10 may communicate with one or more patients 30 and one or more communicants 40 through PSTN 23, Internet 22, and/or a Wireless WAN 21. “PSTN” stands for “Public Switch Telephone Network”. “Wireless WAN” 23 includes public pager networks and public GSM/CDMA cellular networks.

The patient 30 may use the SOS device 31, 32 which is optional and phone 33, fax 35, browser 34 etc. to communicate with the SOS 10.

Communicants 30 may use phone 42, fax 43, email 41 etc to receive alert messages from the SOS 10.

The SOS 10 includes a data store 11, a reporting server 12, an application server 13, a web server 14, a telecommunication server 15 and an SOS engine 16.

The web server 14 functions as an input interface for web-related activities. The telecommunication server 16 functions as an interface of the SOS 10 to different telecommunications input and output channels. The SOS devices, PDAs, and Web Browsers communicate with the SOS 10 through the web server 14. Before reaching the web server 14, RF signals generated by the SOS devices are converted to http messages through a gateway 20 provided by a wireless service provider. Then, the message is sent to the SOS through a web server. Telephones, faxes, and pagers communicate with the SOS 10 through the telecommunication server 15.

The software components in the application server 13 provide the business logic layer. The business logic layer is used to process the messages from input channels and to provide Internet and telephony clients with access to data store. This layer has direct access to the data store 11. After received, messages are processed within this layer and appropriate information is stored in or retrieved from the data store 11.

The SOS engine 16 is a constant running application. It monitors the parameters or thresholds in the data store 11 through the web server 14 and the application server 13. It also monitors the messages of remote SOS devices in data store. Based on information received, the SOS engine decides when and to whom alert or reminder messages are sent. Messages are delegated to the communication server for execution.

The data store 11 is used to store persistent information entered by users and SOS devices. A commercially available RDBMS is used for this data store 11.

The SOS 10 also has a reporting capability. The reporting server 12 provides various reports, including SOS device activity histories. For example, a report of when a patient took his/her medications may be generated. A user may request a specific report through email, web browser, fax, and/or telephone. After the request is processed within the application server 13, the related information is retrieved from the data store 11. Thereafter, a report is generated and presented to the user through the web server, fax, or email. Users can also request the automatic receipt of reports at specified time intervals (e.g., day, week, month, quarter, and year) by fax or email.

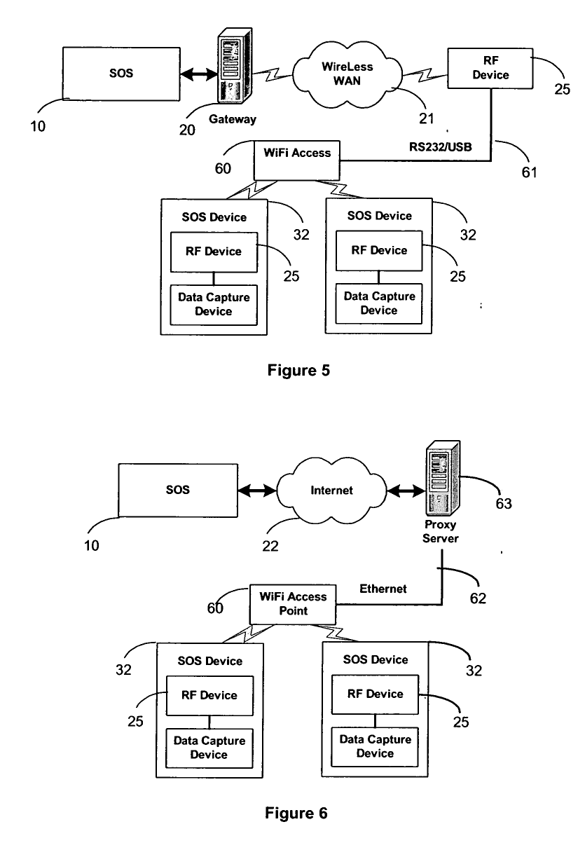

An SOS device 31, 32 includes, in one embodiment, an RF device 25 and a data capture device 27 as shown in FIG. 2. The detailed system diagram of the RF device 25 is shown in FIG. 3. There are two types of RF devices. The first type of RF device uses pagers or cellular protocols in a transceiver 55 to send data to the wireless WAN. The second type of RF device uses a WiFi protocol in the transceiver 55 to route data within a wireless or wired LAN environment. The SOS device using the first type of RF device is labeled 31, FIG. 4. The SOS device using the second type of RF device is labeled 32, FIG. 5. The function of the two RF devices are the same even though their hardware and/or protocols may differ.

An RF device 25 of the SOS device in FIG. 3 includes remote, non-command (mechanical), command (mechanical) and data (electrical) interfaces 50, 51 and 52 to interface with data capture devices 27. Examples (without way of limitation) of data capture devices include, but are not limited to, containers, RFID readers, blood pressure monitors, blood glucose devices, scales, temperature reading devices, blood oxygen level sensors and EKG monitors. The data capture device 27 captures patient-related activities (e.g. use of the device) and data. After the data is captured, it is sent to the SOS 10 by the RF component of the SOS device through a wireless LAN/WAN, traditional wired LAN/WAN, or any other possible network and technology topology.

The mechanical interfaces 50 may be a button, several buttons, or contacts (e.g., two plates that either make or break a contact during the routine use of the SOS device 31, 32 by the user, switches, relays or the like). The mechanical interfaces 50 are used as either non-command interfaces or command interfaces and are triggered upon an event such as the opening of a container (for medication, for example). Examples of other events include pushing a help button, taking a blood pressure, measuring blood glucose or blood oxygenation levels or taking a weight. If the SOS 10 does not receive certain data (e.g., a blood glucose reading) at the predetermined time, the SOS 10 may send a reminder to the SOS device 31, 32. For example, the reminder may indicate that the patient has missed a blood glucose test, blood pressure check, and/or taking their medication and alert the patient to do so as soon as possible.

The alert help interfaces or alert help triggers 51 are similar to the mechanical interfaces 50. However in contrast, the alert help interfaces 51 are used as command interfaces only. A user uses this interface to specifically ask for assistance. This command triggers the SOS device 31, 32 to send out an event message.

The electrical interfaces 52 are preferably RS232/USB or similar type interfaces that are used to read/write data/instruction from/to the data capture device 27. A real time clock 56 (hereinafter “RTC”) may be provided in the RF device to lower the power method of waking the SOS device during periods of inactivity (such as, for example, for heart beat messages) and to be used as a long duration timer. The RTC 56 is backed up with a coin cell lithium or similar battery (not shown). When the RTC 56 alarms, the wake event will latch the input MOSFET ON so the RF device 31/32 can boot. Communication with the RTC 56 is established through a tri-state buffer to allow the RF device 31/32 to multiplex outputs to communicate with both an input multiplexer and the RTC 56. The microprocessor is the central processor unit of the RF device. The RAM is the memory used for the device. The application code of the device is stored in flash.

Radio Frequency Identification Systems (hereinafter “RFID Systems”) are commercially available. A basic RFID system includes an RF reader, antenna, transceiver and decoder, and RF tags electronically programmed with unique information.

In one embodiment, a commercially available Series 2000 RF reader with an RS232 serial communication interface and RF tags from Texas Instruments are used.

The SOS device uses an RFID reader as a data capture device 27 and connects with an RF device 25 through the RS232/USB interface. The RFID reader is used to track RFID tags on patients or important documents to provide better patient and document management and monitoring. When a patient, document or similar object to which an RFID tag is attached moves out of an area or enters a restricted area, the RF reader detects the RFID tag and an event message is sent out by the RF device 25 to the SOS 10.

The SOS device generates the following messages: 1) event messages generated by events such as opening a container, health data input, or triggering of health alerts; and 2) heartbeat messages generated periodically by the SOS device to notify the SOS 10 of the activation status of the SOS device. The heartbeat message has message type equal to zero. The event message has different message types. They are application-specific.

In one embodiment, the event and heartbeat messages have the following format:

| Unique SOS Device Id | Message Type | Data (Optional) |
|---|---|---|
| Service provider | 1 byte | Message type specific |
| specific | | |

Event messages may be further grouped into different categories. The categories include device used event, alert help event, test data event, device alive event etc. Each event group has a unique message type in the message format, which is described herein. For each event message category, the data field in the message format will be different. For example, the data field for test data event will include a measurement and a personal identification. The data field for the device use event will be empty. Therefore, the data field in the message format is optional and message type specific.

The SOS devices may be used independently or in any combination for a given network infrastructure. The following are some examples for how the SOS devices might be used. FIG. 4 is one example utilizing an SOS device in a remote location having no WAN connection. In this example, the RF component 25 of the SOS device is of the type that uses a pager or cellular protocol. The RF component 25 transmits data to the SOS 10 over the wireless WAN 21.

FIG. 5 is a second example utilizing multiple SOS devices in a remote location with no WAN connection. In this case, the RF component 25 of each SOS device 32 uses a WiFi protocol. The RF component 25 of the SOS device 32 sends data to a single WiFi access point 60. The WiFi access point 60 then passes this data to a first type RF component 25 of an SOS device that uses pager or cellular protocol via an RS-232/USB interface 61. The remote RF component 25 then transmits data to the SOS 10 over the wireless WAN 21. In this example, the WiFi access point 60 works as a wireless serial device concentrator.

FIG. 6 is a third example of a topology of the present invention utilizing multiple SOS devices 32 in a corporate or institutional environment, such as a nursing home, hospital or the like. The corporation or institution has an existing connection to the Internet 22. In order for SOS devices 32 to send data to the SOS system 10 over the Internet 22, a proxy server 63, and one WiFi access point 60 are needed, . In this case, the second type of RF component of the SOS device 32 is used. The RF components use a WiFi protocol. Connected behind the WiFi access point 60, SOS devices 32 become IP nodes. The WiFi access point 60 is furnished for the SOS devices to belong to a wireless network. This topology gives users a wireless RS-232 concentrator over Ethernet 62 and IP. The proxy server 63 receives data from the WiFi access point 60 over Ethernet 62 and routes the data through an existing Internet connection to the SOS system 10.

In one embodiment of the SOS device, the SOS device is a medication dispenser having a wireless circuit, (i.e. the RF device 25) embedded within the medication dispenser. When the cover is opened (causing the data capture device to generate a trigger), an RF signal is sent to the SOS 10. If a patient does not take their medication (i.e. does not open the dispenser at a specified time stored in the SOS 10), the SOS 10 sends out reminder messages to the patient (the patient may be called, paged, faxed or the like, or the SOS device may be triggered to emit a reminder alert), and/or alert messages are sent to medical professionals, caregivers or concerned others via the recipient's preferred communication channels (e.g. telephone, fax, email, pager). The SOS 10 gathers and reports dispenser usage for ongoing monitoring of medication compliance. A patient may also press a “help” button on the medication dispenser or other device to request assistance from medical professionals, caregivers, or concerned others.

In the case where the SOS device is a container (e.g., weekly medication box), the RF device of the SOS device is typically embedded within the container. Whenever the container is opened, a triggering mechanism is activated and an RF signal is sent out by the SOS device. There are a number of embodiments for the container. For example, there could be one container with a cover. The container has a number of cells, wherein each cell holds one or more kinds of medication for one dosage for a patient. When the cover is opened, the RF signal is triggered. The signal contains the unique SOS device identification.

In another embodiment, the medication container has a number of cells, and each cell has its own cover. Each cover is mapped to one of more mechanical triggers 50. Each cell is used to hold one kind of medication for a patient. When the cover is opened to one of the cells, an RF signal is generated. The signal contains the unique SOS device identification and the identification of the cell that was opened. The cell identification is used for specific medication identification.

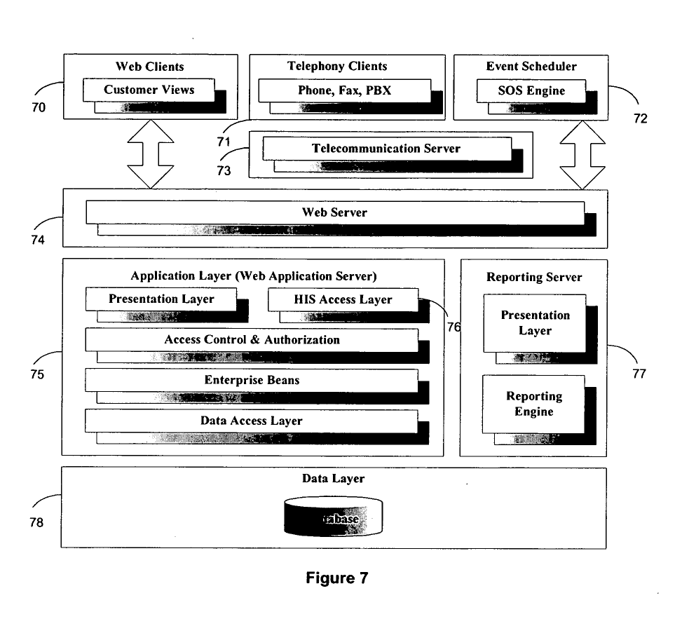

The software architecture of the SOS system is shown in FIG. 7. The SOS, like many large object-oriented systems, is logically structured into layers and partitions. The SOS is represented in vertical tiers. Each vertical layer is decomposed into cohesive units.

The data layer 78 utilizes a commercially available RDBMS such as an Oracle or Sequel Server. The data layer 78 hosts the persistent data related to user information and schedules about patient self-care tasks. The data layer 78 functions as a knowledge base about users and patients.

The application layer 75 provides the SOS with a business rule repository and transaction routing function. The application layer 75 is used by web clients 70, telephony clients 71, and the SOS Engine 72. The business rules include who should be contacted and under what conditions, which communication channels to use when the primary channel is not available etc. This layer processes clients' messages, such as creating/modifying/deleting patients, scheduling data and communicants' contact information etc. The layer provides web clients 70, telephony clients 71 and the SOS engine 72 centralized access to the data store. This layer also provides HIS and HL7 interfaces 76 for integration with hospital information systems. This layer is implemented by a commercially available application such as Weblogic, Jrun, Timecat etc. The web server 74 provides an interface for web clients to access the application layer.

The telecommunication server 73 consists of standard telephony components, primarily Intel dialogic boards and telephony management software. The Telephony components provide telephony operations: 1)Incoming call detection, 2) DTMF detection, 3) Pre-recorded message playback, 4) call transfer, 5) audio message recording, 6) text-to-speech generation, 7) speech recognition and verification, 8) fax, 9) outgoing call generation and 10) audio broadcasting. These components can directly interface with a PSTN 23 or integrate with PBXs. The telephony management software represents the call logic portion. It includes the IVR application and outgoing call generation software. It also serves as the interface between the telephony components and the application layer. For example, when a patient uses a phone to type in their user id and password, the telephony software takes the DTMF tone from a telephony component and sends it to the application server for verification. The telecommunication server communicates with the web server 74 and application layer 75 over VoiceXML.

The SOS engine is an event scheduler. It runs as a demon. It periodically checks with the data store through the web server/application server for events needing attention and contacts the telecommunication server for actions such as outgoing call generation etc.

The reporting server 77 is a commercially available server such as Crystal Reports. The reporting server accesses the data store 83 through data access components provide by the server. The reporting server 77 has a presentation layer that can be customized to generate different views to meet different report navigation needs.

The reporting server 77 and the application server 75 in the previous sections can be integrated into the same web server as shown in FIG. 7.

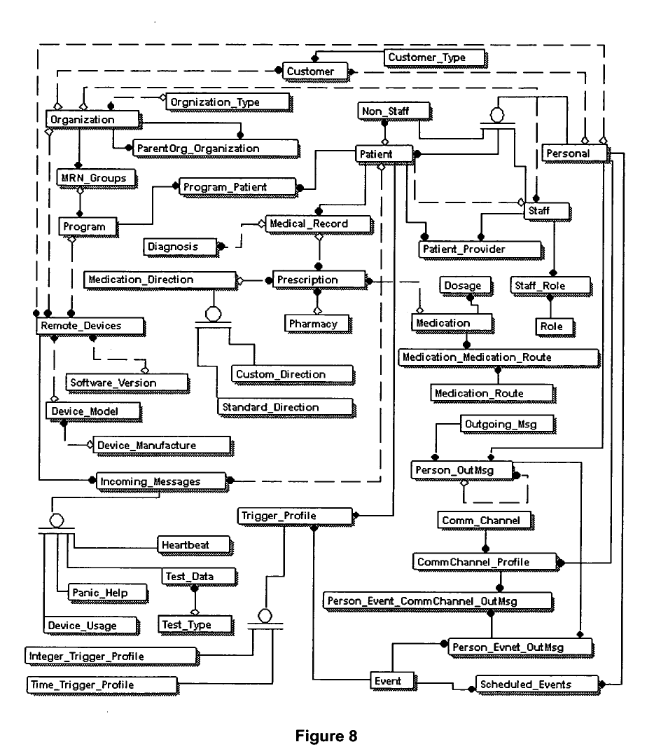

A database schema is created to model the user information as shown in FIG. 8. The schema consists of various tables and relationships. It uses IDEF1X (Integration Definition for Information Modeling) notation although this is not a limitation of the invention but merely the preferred embodiment. The following entities are modeled in, the data schema: 1) Different health care organizations such as an insurance companies, pharmacies, Healthcare Delivery Organizations, Groups of programs that use a unique medical record “Medical Record Group”), medical practices, etc., which may use the SOS 10; 2) Persons involved in different scenarios of reminders, alerts and monitoring including patients, organization staff, healthcare providers as well as patient relatives and other significant others; 3) Remote SOS devices and various messages received by the SOS from these devises; 4) Scheduled reminder, alert and monitoring events, event triggers and event handlers, etc.; 5) Notification communication channels and outgoing messages; 6) Billing/account management; and 7) Technical/customer support.

The details of the entities such as the user activity log, customer accounts, customer statements, as well as system authorization and access control, etc. are important but not relevant to this invention. Therefore, they are omitted from the data schema in FIG. 8. The relationships among the entities are also developed in the data schema. Entity and referential integrity constraints are enforced in the database schema.

The Remote_Devices table is used for SOS devices. An organization or a person such as a patient can own one or more SOS devices. The Organization has one-to-many relationship to the Remote_Devices table. The Personal table also has a one-to-many relationship to the Remote_Devices table.

The Incoming_Messages table is used to store messages from the SOS devices. One device can send out unlimited messages. The Remote_Devices table has a one-to-many relationship with the Incoming_Messages table. There can be many different types of incoming messages. In the schema, only a few sub-type messages are modeled. They are heartbeat messages, device usage messages (i.e., when the device is used), panic help alert messages and patient test data messages.

Users may specify communication channel priorities for message receipt. If a user chooses multiple channels as priority “1”, then the SOS 10 will send multiple simultaneous messages to those channels as specified.

A user can pre-store text and voice messages in the system or use default outgoing messages for alerts and reminders. The message id and paths to the default message are stored in the Outgoing_Msg table. The information related to personal messages is stored in the Person_OutMsg table. A user can compose a sequence of messages into one message. A recursive relationship to the Person_OutMsg table is used to model this feature.

To receive alert and reminder messages, a user can choose from a list of communication channels defined in Comm_Channel. The CommChannel_Profile table is used to store the communication channels a user selected and the priority of each channel.

Events defined in the Event table include reminders for appointments and to take medications as well as to perform other self-care events. Users can enter a schedule in the Scheduled_Event table for an event defined in Event table. Outgoing messages are generated based on time parameters defined in the Scheduled_Event table. A user can also enter a trigger for an event in the Trigger_Profile table. The trigger could be receiving a device trigger message from an SOS device or an SOS test result that falls outside of predefined result thresholds. Once the trigger is fired, the SOS 10 sends out outgoing messages to the people involved for a given event. The relationships among event, personal, communication channels and outgoing messages are shown in FIG. 8.

The SOS 10 assists patients in performing self-care tasks. The SOS 10 sends alerts and reminder messages to message recipients and monitors a patient's behavior (e.g., self-care event completed). This system and apparatus may also be used in many other systems for alerting, reminding, and monitoring. Moreover, there are many different ways of implementing the remote sensors, database schema and communication channels in addition to the preferred embodiment of the system described herein. Further, the techniques for sending messages and receiving responses using a variety of different networks will work for networks other than those used in the preferred embodiment.

For all of the foregoing reasons, the previous description is to be regarded as being in all respects exemplary and not restrictive. Modifications and substitutions by one of ordinary skill in the art are considered to be within the scope of the present invention, which is not to be limited except by the following claims.

What is claimed is:

1. An SOS system for monitoring and reporting self-care compliance and health status data, for prompting, and/or reminding a patient of self-care events, and for generating alerts and messages if self-care events are not completed by the patient, to report abnormal health status data to healthcare providers and other concerned others, and to communicate emergency requests for help from the patient to health care providers or concerned other, the system comprising:
an SOS device, for receiving incoming self-care event reminder alerts, for generating event messages indicating the completion of the self-care event, for sending health status data and for sending alert messages; and
wherein the system receives, processes and stores the data, alert and event messages from the SOS device.

2. The system according to claim 1, wherein responsive to said processing, the SOS system either sends an alert message to another person signaling that the patient has not satisfied the self-care event by a specified predetermined time if the SOS device fails to send an event message indicating the completion of a self-care event, or sends an alert message to others signaling that the patient has satisfied the self-care event if the SOS device does send an event message indicating the completion of the self-care event.

3. The system according to claim 2, wherein the predetermined time is designated by at least one user selected from the group consisting of the patient, another person, one or more concerned parties such as a family member or significant other, disease management specialists, healthcare insurance provider, and health care provider.

4. The system according to claim 3, wherein the SOS device includes an RF device.

5. The system according to claim 4, wherein the SOS device includes a non-command user interface, for automatically generating the event message indicating the completion of a self-care event.

6. The system according to claim 4, wherein the SOS device includes a command user interface, for generating said alert messages.

7. The system according to claim 6, wherein the command user interface causes the system to send a message to a third party requesting an immediate, emergency response, and wherein the message has an identification of the patient and a unique panic code.

8. The system according to claim 7, wherein the SOS device further includes a data input device, for sending Health status data to the system.

9. The system according to claim 1, wherein the system sends out said self-care event reminder alerts to a patient by way of said reminder alert message to the SOS device.

10. The system according to claim 4, wherein the RF device is operatively connected to one or more of a pager, or a cellular wireless WAN and one or more WiFi devices on a local area network.

11. The system according to claim 10, wherein the RF device has a unique identification code and sends a message to the system to notify the system of the aliveness of the device.

12. The system according to claim 11, wherein the RF device includes an electronic interface and a mechanical interface.

13. The system according to claim 12, wherein the electronic interface includes an RS232 serial communication interface or USB interface and the mechanical interface device includes a mechanical trigger.

14. The system according to claim 13, wherein the SOS device includes an electronic interface device for reading and writing data and sending out messages when either the mechanical trigger is activated, when health status data is collected, or when the patient presses and emergency help button.

15. The system according to claim 10, wherein the SOS device communicates with the system by means of a public wireless WAN.

16. The system according to claim 13, wherein the RF device communicates with the WiFi access devices via the RS232/USB interface, wherein the WiFi access devices communicate with multiple SOS devices, and wherein the RF device communicates with the system by means of at least one of a public wireless LAN and a wired LAN.

17. The system according to claim 10, wherein the WiFi access device communicates with multiple SOS devices and the WiFi access point is operatively connected to a proxy server, and wherein the proxy server operatively connects to the system by means of an Internet connection.

18. The system according to claim 1, further including a data capture device comprising a medication dispenser with at least one cover.

19. The system according to claim 1, further including a data capture device including a medical device container having at least one cover.

20. The system according to claim 18, wherein the mechanical trigger is activated when the cover of the medication dispenser is opened, thereby signaling and communicating the completion of the self-care event from the SOS device to the system.

21. The system according to claim 19, wherein the mechanical trigger is activated when the cover of the medical device container is opened, thereby signaling and communicating the completion of the self-care event from the SOS device to system.

22. The system according to claim 18, further including cells covered by the covers and wherein the mechanical trigger is activated when one or more of multiple covers of the medication dispenser are opened, thereby communicating from the SOS device to the system the completion of the self-care event and an identification of the specific covers and cells that were opened.

23. The system according to claim 19, further including a plurality of storage cells covered by the covers and a plurality of mechanical triggers, and wherein a mechanical trigger is activated when each of the multiple covers of the medical device container are opened, thereby signaling and communicating the completion of the self-care event for each cell and an identification of the specific covers and cells that were open from the SOS device to the system.

24. The system according to claim 22, wherein the data capture device includes an RFID system and wherein the RFID system is integrated into the SOS device.

25. The system according to claim 23, wherein the data capture device includes an RFID system and wherein the RFID system is integrated into the SOS device.

26. The system according to claim 10, wherein the system includes a database containing information including personal profile, communication channels, incoming messages from the SOS device, outgoing alert and reminder message files, predefined triggering events, account management files, and customer and technical support information files.

27. The system according to claim 26, wherein the database provides information for the system to determine when, how and to whom alert messages and reminder message are sent.

28. The system according to claim 10, wherein the system communicates with the SOS device by means of at least one of a public wireless WAN, Internet and a LAN.

29. The system according to claim 10, wherein the system communicates with a Hospital Information System or other third party systems via HL7 and XML.

30. The system according to claim 10, further including a plurality of interfaces including a public switched telephone network interface.

31. The system according to claim 10, further including a plurality of interfaces including a public wireless WAN interface.

32. The system according to claim 10, further including a plurality of interfaces including an Internet interface.

33. The system according to claim 10, further including a plurality of input interfaces selected from the group consisting of an IVR, Fax, web, phone, PDA, email, and remote monitoring devices.

34. The system according to claim 33, further including a plurality of output interfaces selected from the group consisting of Fax, web, phone, email, Wireless PDA, SOS devices, and Pager.

35. A system (SOS) for monitoring and prompting, self-care events, and reporting self-care compliance, emergency help alerts and health status via multiple user-selected channels to others, that notifies the users that the patient either completed or did not complete a self-care event, the system comprising a self-care optimization system having at least one SOS device, wherein the SOS device is preprogrammed to monitor and remind the patient to perform the self-care event and the SOS device is operatively in communication with one or more communicants through the SOS, thereby communicating completed and uncompleted self-care events and health status data to one or more communicants.

36. The system according to claim 35, wherein the SOS includes a reporting server for providing reports of the data collected from and the activities of the SOS device, a data store for storing information gathered by SOS devices, an application server for business rules and access to the data store, an SOS engine for monitoring the parameters in the data store and inputted messages from remote SOS devices and for deciding when and whom to alert, and a telecommunication server for interfacing with telephony input and output channels, IVR, and for outgoing call generation. The telecommunication server is integrated with the web server and the application server through VoiceXML.

37. The system according to claim 36, wherein the input and output channels are selected from the group consisting of a telephone, fax, computer email, SOS device, or pager.

38. The system according to claim 37, wherein the SOS device is selected from the group consisting of a pager, WIFI device, cellular device, multi-channel communication system, fax, email, Web Browser, telephone, pager, and PDA.

39. A system for monitoring health status, the system comprising a self-care optimization system (SOS) having at least one SOS device, wherein at least one SOS device is designed and preprogrammed to obtain at least one health status criterion value and wherein the SOS device is operatively in communication with the SOS system for communicating to said SOS system said health status criterion value.
